(19) United States
(12) Patent Application Publication
(10) Pub. No.: US 2022/0000944 A1
(43) Pub. Date: Jan. 6, 2022

(54) AGENT FOR CONTROLLING SEBACEOUS GLANDS

(71) Applicants: THE RITSUMEIKAN TRUST, Kyoto-shi, Kyoto (JP); NICHINICHI PHARMACEUTICAL CO., LTD., Iga-shi, Mie (JP)

(72) Inventors: Takashi FUJITA, Kusatsu-shi, Shiga (JP); Takashi SHIMADA, Iga-shi, Mie (JP)

(21) Appl. No.: 17/291,325

(22) PCT Filed: Nov. 8, 2019
(86) PCT No.: PCT/JP2019/043999

§ 371 Date: May 5, 2021

(30) Foreign Application Priority Data
Nov. 9, 2018 (JP) 2018-211210

(51) Int. Cl. A61K 35/744; A61K 8/99; A61Q 15/00; A61Q 19/00

(52) U.S. Cl. CPC A61K 35/744; A61Q 19/00; A61Q 15/00; A61K 8/99

(57) ABSTRACT

This invention relates to an external agent for normalizing sebaceous glands, the external agent comprising at least one member selected from the group consisting of cells of lactic acid bacteria belonging to the genus Enterococcus and cell components thereof, or a component that is extracted from cells of lactic acid bacteria belonging to the genus Enterococcus and that is soluble in ethyl acetate.

### TECHNICAL FIELD

The present invention relates to an external agent for normalizing sebaceous glands.

### BACKGROUND ART

Acne (acne vulgaris) is a chronic inflammatory disease of the pilosebaceous unit, and its significant pathophysiology is considered to include increased secretion of sebum due to the sebaceous gland stimulation effect of androgens, pore clogging due to abnormal keratinization in infundibular hair follicles, and the presence of organisms in infundibular hair follicles. Other internal and external factors, including genetic factors, age, dietary factors, environmental factors such as temperature and humidity, mechanical stimuli, stress, cosmetics, and drugs, are also considered to be involved.

Lactic acid bacteria belonging to the genus Enterococcus are known to have various effects on living organisms.

For example, an Enterococcus faecalis NF-1011 strain has been reported to have the following effects: inhibition of blood pressure increase and prevention of cardiac hypertrophy (Patent Literature 1), immunostimulation effects (Patent Literature 2), enhancement of interferon production (Patent Literature 3), protection against infection (Patent Literature 4), enhancement of anti-cancer (Patent Literature 5), reduction of anti-cancer drug toxicity (Patent Literature 6), and activation of biological antioxidant capacity (Patent Literature 7).

However, Patent Literature 1 to 7 basically disclose oral administration of lactic acid bacteria, and do not assume their use as external agents.

### CITATION LIST

### Patent Literature

PTL 1: JP1993-201871A

PTL 2: JP1996-99887A

PTL 3: JP1996-259450A

PTL 4: JP1996-283166A

PTL 5: JP1996-295631A

PTL 6: JP1997-48733A

PTL 7: JP2017-1961A

### SUMMARY OF INVENTION

### Technical Problem

The present invention aims to provide an external agent that has an excellent effect of normalizing sebaceous glands.

### Solution to Problem

To achieve the above object, the present inventors conducted extensive research. As a result, they found that in sebaceous gland cells, the cells of an Enterococcus faecalis NF-1011 strain and cell components thereof inhibit sebum production induced by Cutibacterium acnes when present, and they stimulate sebaceous glands to produce sebum in the absence of Cutibacterium acnes.

The present invention was accomplished as a result of further research based on the above findings. The present invention provides the following external agent for normalizing sebaceous glands.

Item 1. An external agent for normalizing sebaceous glands, comprising at least one member selected from the group consisting of cells of lactic acid bacteria belonging to the genus Enterococcus and cell components thereof, or a component that is extracted from cells of lactic acid bacteria belonging to the genus Enterococcus and that is soluble in ethyl acetate.
Item 2. The external agent according to Item 1, wherein the lactic acid bacteria belonging to the genus Enterococcus are Enterococcus faecalis.
Item 3. The external agent according to Item 1 or 2, wherein the lactic acid bacteria belonging to the genus Enterococcus are Enterococcus faecalis NF-1011 strain (FERN BP-10902).
Item 4. The external agent according to any one of Items 1 to 3, wherein the cells are dead cells.
Item 5. The external agent according to any one of Items 1 to 4, wherein the cell component of lactic acid bacteria is a component obtained by subjecting the lactic acid bacteria to a lytic enzyme treatment and a heat treatment.
Item 6. The external agent according to any one of Items 1 to 5, wherein the external agent is used for promoting or inhibiting sebum secretion, preventing or improving dry skin, preventing or improving oily skin, moisturizing, anti-acne, or inhibiting body odor.
Item 7. A method for normalizing sebaceous glands, comprising the step of applying at least one member selected from the group consisting of cells of lactic acid bacteria belonging to the genus Enterococcus and cell components thereof, or a component that is extracted from cells of lactic acid bacteria belonging to the genus Enterococcus and that is soluble in ethyl acetate to the skin of a mammal in need of sebaceous glands normalization.
Item 8. The method according to Item 7, wherein the lactic acid bacteria belonging to the genus Enterococcus are Enterococcus faecalis.
Item 9. The method according to Item 7 or 8, wherein the lactic acid bacteria belonging to the genus Enterococcus are Enterococcus faecalis NF-1011 strain (FERN BP-10902).
Item 10. The method according to any one of Items 7 to 9, wherein the cells are dead cells.
Item 11. The method according to any one of Items 7 to 10, wherein the cell component of lactic acid bacteria is a component obtained by subjecting the lactic acid bacteria to a lytic enzyme treatment and a heat treatment.
Item 12. Use of at least one member selected from the group consisting of cells of lactic acid bacteria belonging to the genus Enterococcus and cell components thereof, or a component that is extracted from cells of lactic acid bacteria belonging to the genus Enterococcus and that is soluble in ethyl acetate in the production of an external agent for normalizing sebaceous glands.
Item 13. The use according to Item 12, wherein the lactic acid bacteria belonging to the genus Enterococcus are Enterococcus faecalis.
Item 14. The use according to Item 12 or 13, wherein the lactic acid bacteria belonging to the genus Enterococcus are Enterococcus faecalis NF-1011 strain (FERM BP-10902).
Item 15. The use according to any one of Items 12 to 14, wherein the cells are dead cells.
Item 16. The use according to any one of Items 12 to 15, wherein the cell component of lactic acid bacteria is a component obtained by subjecting the lactic acid bacteria to a lytic enzyme treatment and a heat treatment.

### Advantageous Effects of Invention

Cells of lactic acid bacteria belonging to the genus Enterococcus and cell components thereof stimulate sebaceous glands to produce sebum in sebaceous gland cells under normal conditions, while they inhibit sebum production when an abnormally high amount of sebum is produced. Accordingly, the cells and cell components are useful as active ingredients of external agents for normalizing sebaceous glands, particularly external agents for promoting or inhibiting sebum secretion, preventing or improving dry skin, preventing or improving oily skin, moisturizing, anti-acne, or inhibiting body odor.

### BRIEF DESCRIPTION OF DRAWINGS

FIG. 1 shows photographs indicating the results of SZ-95 sebaceous gland cells with LFK or FK-23 in the presence or absence of Cutibacterium acnes in Test Example 1 observed with fluorescent microscopy. The bar is 50 μm.

FIG. 2 is a chart showing the fraction process of FK-23 in Test Example 2.

FIG. 3 shows photographs indicating the results of SZ-95 sebaceous gland cells with the fractions of FK-23 in the presence or absence of Cutibacterium acnes in Test Example 2 observed with fluorescent microscopy.

### DESCRIPTION OF EMBODIMENTS

The embodiments of the present invention are detailed below.

In the present specification, the term “comprise” includes the meanings of essentially consisting of and consisting of.

The external agent for normalizing sebaceous glands according to the present invention comprises at least one member selected from the group consisting of cells of lactic acid bacteria belonging to the genus Enterococcus and cell components thereof, or a component that is extracted from cells of lactic acid bacteria belonging to the genus Enterococcus and that is soluble in ethyl acetate.

The lactic acid bacteria belonging to the genus Enterococcus are not particularly limited. Examples include Enterococcus faecalis, Enterococcus faecium, Enterococcus avium, Enterococcus casseliflavus, Enterococcus gallinarum, Enterococcus flavescens, and the like. Of these, Enterococcus faecalis, Enterococcus faecium, or the like are preferred, and Enterococcus faecalis is more preferred. Of Enterococcus faecalis, an Enterococcus faecalis NF-1011 strain, which is isolated from feces of a healthy individual, is preferred. An Enterococcus faecalis NF-1011 strain was deposited in the National Institute of Advanced Industrial Science and Technology, Patent Organism Depositary Center (Central No. 6, 1-1-1 Higashi, Tsukuba, Ibaraki, Japan (postal code 305-8566)), on Oct. 8, 1991, with the accession number FERM P-12564. This strain was then transferred to the international depositary with the accession number FERM BP-10902. In April 2012, the National Institute of Advanced Industrial Science and Technology, Patent Organism Depositary Center was consolidated into the National Institute of Technology and Evaluation (NITE), Patent Microorganisms Depositary, and the microorganism depository operation has been succeeded by the National Institute of Technology and Evaluation Biotechnology Center, International Patent Organism Depositary (NITE-IPOD) (#120, 2-5-8 Kazusakamatari, Kisarazu-shi, Chiba 292-0818, Japan).

The cells of lactic acid bacteria belonging to the genus Enterococcus are not particularly limited as long as they are the entire structure of the lactic acid bacteria belonging to the genus Enterococcus. The cells may be viable cells or dead cells. The cells may be a dried product such as a lyophilized product. The viable cells of lactic acid bacteria belonging to the genus Enterococcus can be ordered from national or international distribution organizations, such as ATCC, IFO, and JCM, or can be isolated from an organism.

Since viable cells can be easily produced in a large quantity by culture, use of cultured viable cells is economical with low production costs. Viable cells of lactic acid bacteria belonging to the genus Enterococcus can be also proliferated by culture according to a known method. For example, a large amount of viable cells can be obtained by seeding the lactic acid bacteria in an appropriate amount of sterile Rogosa liquid medium, statically culturing the bacteria at 35 to 37° C. for 10 to 16 hours in an aerobic manner to obtain a preliminary culture liquid, adding the preliminary culture liquid to a large quantity of sterile Rogosa liquid medium to perform static culture in the same manner. When viable cells are used, a culture liquid itself may be used, or a solid of the culture liquid (e.g., a precipitate obtained by precipitating viable cells in the culture liquid by centrifugation and the like, and optionally washing with a physiological saline solution) can be used, or a suspension of the solid (e.g., a suspension obtained by suspending viable cells in an isotonic solution such as a physiological saline solution) can be used.

The dead cells of lactic acid bacteria belonging to the genus Enterococcus are not particularly limited. For example, heat-treated viable cells can be used. The temperature of the heat treatment is not particularly limited as long as it is 100° C. or higher. It is preferably a temperature at which an autoclave treatment can be performed (e.g., 110 to 125° C.). The heat treatment time is, for example, 1 minute or more, preferably 5 to 20 minutes, and more preferably about 5 to 15 minutes.

The cells of a Lactobacillus Enterococcus faecalis NF-1011 strain are commercially available, for example, as FK-23 (trademark) from Nichinichi Pharmaceutical Co., Ltd.

In the present invention, the term “cell component of lactic acid bacteria” means a component that is released outside the cells as a result of the destruction of the cell wall of lactic acid bacteria.

The cell component of lactic acid bacteria is not particularly limited. It is, for example, a component in which the cell wall of viable cells is destroyed. The cell wall to be destroyed may be all or part of the cell wall of viable cells. Examples of the cell wall-destroying method include heat treatment, treatment by physical force, treatment with a lytic enzyme, and combinations thereof. Of these, a method comprising treatment with a lytic enzyme is preferred. A method comprising (a) treatment with a lytic enzyme and (b) at least one treatment selected from the group consisting of heat treatment and treatment by physical force (preferably heat treatment) is more preferred; and a method in which (b) at least one treatment selected from the group consisting of heat treatment and treatment by physical force (preferably heat treatment) is performed after (a) treatment with a lytic enzyme is even more preferred.

The temperature of the heat treatment is not particularly limited as long as it is 100° C. or higher. It is preferably a temperature at which autoclave treatment can be performed (e.g., 110 to 125° C.). The heat treatment time is not particularly limited as long as all or part of the cell wall can be destroyed, and can be suitably set according to the temperature of the heat treatment. The heat treatment time is, for example, 1 minute or more, preferably 5 to 20 minutes, and more preferably about 5 to 15 minutes.

The method of treatment by physical force is not particularly limited as long as all or part of the cell wall can be destroyed. Examples include an ultrasonic treatment, French press, and the like.

The enzyme used for the treatment with a lytic enzyme is not particularly limited as long as it can destroy all or part of the cell wall. Various enzymes generally used for lysing bacteria can be used. Examples include lysozyme, actinase, zymolyse, chitarase, mutanosylin, and acromopeptidase. Of these, lysozyme is preferred. One kind of lytic enzyme may be used, and two or more kinds of lytic enzymes may be used in combination.

Conditions for treatment with a lytic enzyme can be suitably determined according to the kind of lytic enzyme, the amount of a lysis target (viable cells), and the like. For example, the lytic enzyme may be added to a viable cell suspension so that the final concentration is 0.01 to 1 mg/mL, and a treatment is performed at 30 to 40° C. for 1 to 10 hours.

The cell component of lactic acid bacteria is not particularly limited as long as it is a component constituting the cells of lactic acid bacteria. The cell component is preferably a water-soluble component. The water-soluble component is obtained, for example, by removing a solid by centrifugation or the like from lactic acid bacteria whose cell wall has been destroyed.

The cell component of Lactobacillus Enterococcus faecalis NF-1011 strain is commercially available, for example, as LFK (trademark) from Nichinichi Pharmaceutical Co., Ltd.

As the cells of lactic acid bacteria belonging to the genus Enterococcus and the cell component thereof, a component that is extracted from cells of lactic acid bacteria belonging to the genus Enterococcus and that is soluble in ethyl acetate is preferred. As shown in the Examples described below, of the components extracted from the cells of lactic acid bacteria belonging to the genus Enterococcus, those that are soluble in ethyl acetate have a sebaceous gland normalizing effect.

Since the external agent of the present invention has an effect of normalizing sebaceous glands due to the cells of lactic acid bacteria belonging to the genus Enterococcus and the cell component thereof, it is suitably used as an external agent for promoting or inhibiting sebum secretion, preventing or improving dry skin, preventing or improving oily skin, moisturizing, anti-acne, or inhibiting body odor. The external agent of the present invention includes an external drug and a cosmetic. The cosmetic also includes a quasi-drug. The external agent of the present invention is applied to the skin (including the scalp) of mammals, including humans.

In preparing a drug, the cells of lactic acid bacteria and the cell component thereof are prepared, together with a known ingredient, into the form of an external solid preparation, an external liquid preparation, a spray, an ointment, a cream, a gel, a paste, and the like, thus obtaining an external preparation.

The drug may include one or more kinds of known additives used for an external agent, i.e., one or more kinds of known additives selected from antibacterial agents, coolants, emulsifiers, oils, antioxidants, surfactants, fragrances, UV absorbers, dyes, ethanol, water, moisturizers, thickeners, solubilizers, gelling agents, and the like.

The proportion of cells of lactic acid bacteria or cell component thereof contained in the drug is not particularly limited. The proportion is for example, 0.01 to 99 mass %.

Cosmetics can be formulated in various forms, including an aqueous solution, solubilization, emulsion, oil-liquid, powder, gel, ointment, aerosol, water-oil two-layer form, and water-oil-powder three-layer form.

The cosmetics can be used for any application. Examples include basic cosmetics, such as facial washes, lotions, milky lotions, creams, gels, essences, serums, packs, and masks; make-up, such as foundation, lipstick, rouge, eye shadow, eyeliner, and mascara. Other examples include facial cleanser, massage agents, cleansing agents, after-shave lotion, pre-shave lotion, shaving cream, body soap, soap, shampoo, rinse, hair treatment, hair styling products, hair tonic, antiperspirant, bath powder, and the like.

In addition to the cells of lactic acid bacteria or cell component thereof, the cosmetic suitably contains an ingredient that is generally used in cosmetics, as necessary. Examples include a whitening agent, moisturizer, antioxidant, oily component, UV absorber, surfactant, thickener, alcohol, powdery component, colorant, aqueous component, water, various skin nutrient, or the like.

The proportion of the cells of lactic acid bacteria or cell component thereof in the cosmetic is not particularly limited. For example, the proportion is 0.01 to 99 mass %.

As shown in the Examples below, since the present inventors found that the cells of lactic acid bacteria belonging to the genus Enterococcus and the cell components thereof stimulate sebaceous glands to produce sebum in sebaceous gland cells under normal conditions, while they suppress the production of sebum when an abnormally high amount of sebum is produced, the following effects are expected: promotion or inhibition of sebum secretion, prevention or improvement of dry skin (in the elderly, patients with kidney disease, patients with diabetes, etc.), prevention or improvement of oily skin, moisturization, anti-acne effects, and inhibition of body odor (e.g., axillary odor). Acne and body odor such as axillary odor are caused by excessive sebum secretion.

Accordingly, since the cells of lactic acid bacteria belonging to the genus Enterococcus and the cell component thereof have a significantly high normalization effect (particularly an effect of normalizing sebaceous secretion) in sebaceous gland cells, they are useful as an active ingredient of an external agent for normalizing sebaceous glands, particularly an external agent for promoting or inhibiting sebum secretion, preventing or improving dry skin, preventing or improving oily skin, moisturizing, anti-acne, inhibiting body odor, and the like.

### EXAMPLES

The following examples are given to illustrate the present invention in more detail. However, the present invention is not limited to these examples.

### Test Example 1

### Experimental Method

### 1. Preparation of Cell Sample: FK-23

An Enterococcus faecalis NF-1011 strain was cultured in a liquid medium (glucose: 2%, yeast extract: 2%, peptone: 2%, dipotassium hydrogen phosphate: 4%) at 37° C. for 18 hours. Using a microfiltration membrane, harvesting and washing were performed to collect viable cells. The viable cells were heat-treated at 110° C. for 10 minutes, and then dried by spray drying. The resulting dried dead cells were used as a cell sample (FK-23) in the following experiment.

### 2. Preparation of Cell Sample: LFK

An Enterococcus faecalis NF-1011 strain was inoculated in 10 ml of Rogosa liquid medium and statically cultured (preliminarily cultured) aerobically at 37° C. for 15 hours to obtain a cell solution (seed) having a cell concentration of approximately 10⁹ cells/ml. The cells were inoculated in 10 L of Rogosa liquid medium (cell concentration: 10⁶ cells/ml) and statically cultured aerobically at 37° C. for 16 hours to obtain a cell solution having a viable cell count of approximately 10⁹ cells/ml. The obtained cell solution was harvested by centrifugation (12,000×g for 20 minutes), followed by washing twice with a physiological saline solution (0.85% sodium chloride solution) and suspension in 100 ml of distilled water, thus obtaining a cell suspension. Lysozyme was added to the cell suspension so that the final concentration was 0.1 mg/ml, followed by a treatment at 37° C. for 4 hours. Thereafter, heat treatment at 110° C. for 10 minutes was conducted to obtain treated cells. The resulting treated cells were used as a cell sample (LFK) in the following experiment.

### 3. SZ-95 Sebaceous Gland Cell Differentiation Test

Human sebaceous gland cell strain SZ-95 was imported from the Dessau Medical Center (Germany), and cultured in a Sebomed basal medium (Millipore, Billerica, Mass.) supplemented with 10% FCS (Thermo Fisher Scientific Inc., Yokohama, Japan), 50 IU/ml penicillin and 50 μg/ml streptomycin (Nacalai Tesque, Kyoto, Japan), and 5 ng/ml human EGF (PeproTech GmbH, Hamburg, Germany). The medium was changed every other day, and the cells were sub-cultured at 60 to 70% confluence.

Thereafter, the cells were exposed to LFK and FK-23 in the presence or absence of Cutibacterium acnes extract for 24 hours in a medium without hEGF. SZ-95 cells were seeded in an 8-well chamberslide at a concentration of 5.0×10⁴ cells/well. LFK and FK-23 were individually added at a final concentration of 300 μg/ml, and Cutibacterium acnes was added at 2.0×10⁵ CFU/ml, followed by incubation at 37° C. for 24 hours. After the incubation, the fat-compatible indicator BODIPY was added at a final concentration of 1 μM, and observation was performed using the EVOS FL microimaging system. A similar follow-up test was conducted and reproducibility was confirmed.

### Results

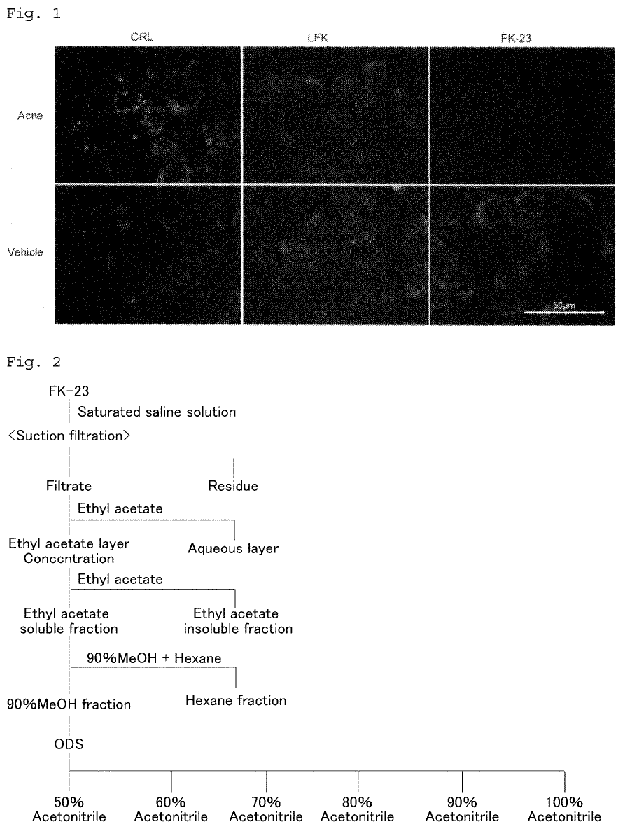

The results are shown in FIG. 1. In the absence of Cutibacterium acnes, LFK and FK-23 stimulated sebaceous glands to produce sebum. In contrast, in the presence of Cutibacterium acnes, LFK and FK-23 inhibited sebum production induced by Cutibacterium acnes.

### Test Example 2

### Experimental Method

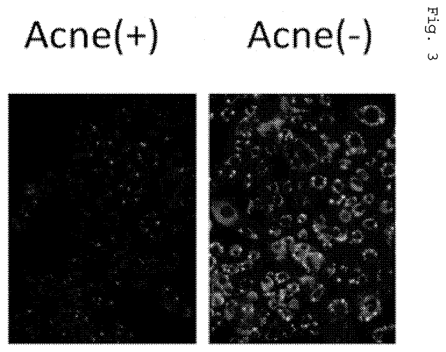

In order to search for a component showing the effects confirmed in Test Example 1 in FK-23, fractionation of FK-23 was performed by the method shown in FIG. 2.

A filtrate obtained by suspending FK-23 prepared in Test Example 1 in a saturated saline solution, and subjecting the suspension to suction filtration was fractioned by solvent partition using ethyl acetate. The resulting ethyl acetate layer was dried and solidified using a decompression evaporator, and then ethyl acetate was added to separate a soluble portion and an insoluble portion. The component soluble in ethyl acetate was fractioned by solvent partition using 90% MeOH and hexane. As a result, the 90% MeOH fraction showed strong activity, and thus purification was performed using a medium-pressure column. As the medium-pressure column, an ODS (SHOKO Purif-Pack (trademark)-EX ODS-50 μm, size: 20, 60×20 mm I.D., produced by Shoko Science Co., Ltd.) was used. Using mixed solvents of acetonitrile and water, elution was conducted stepwise in the order of 50% acetonitrile, 60% acetonitrile, 70% acetonitrile, 80% acetonitrile, 90% acetonitrile, and 100% acetonitrile.

A SZ-95 sebaceous gland cell differentiation test as in Test Example 1 was performed for each of the obtained fractions.

### Results

The results are shown in FIG. 3. FIG. 3 indicates that the 50% acetonitrile fraction attained results similar to those of Test Example 1; accordingly, it was found that the ethyl acetate soluble fraction contained a component exhibiting a sebaceous gland normalization effect.

1. A method for normalizing sebaceous glands, comprising the step of applying at least one member selected from the group consisting of cells of lactic acid bacteria belonging to the genus Enterococcus and cell components thereof to the skin of a mammal in need of sebaceous glands normalization.

2. The method according to claim 1, wherein the lactic acid bacteria belonging to the genus Enterococcus are Enterococcus faecalis.

3. The method according to claim 1, wherein the lactic acid bacteria belonging to the genus Enterococcus are Enterococcus faecalis NF-1011 strain (FERM BP-10902).

4. The method according to claim 1, wherein the cells are dead cells.

5. The method according to claim 1, wherein the cell component of lactic acid bacteria is a component obtained by subjecting the lactic acid bacteria to a lytic enzyme treatment and a heat treatment.

6. The method according to claim 1, wherein the method is a method for promoting or inhibiting sebum secretion, preventing or improving dry skin, preventing or improving oily skin, moisturizing, anti-acne, or inhibiting body odor.

7. A method for normalizing sebaceous glands, comprising the step of applying a component extracted from cells of lactic acid bacteria belonging to the genus Enterococcus, and the component being soluble in ethyl acetate to the skin of a mammal in need of sebaceous glands normalization.

8. The method according to claim 7, wherein the lactic acid bacteria belonging to the genus Enterococcus are Enterococcus faecalis.

9. The method according to claim 7, wherein the lactic acid bacteria belonging to the genus Enterococcus are Enterococcus faecalis NF-1011 strain (FERM BP-10902).

10. The method according to claim 7, wherein the method is a method for promoting or inhibiting sebum secretion, preventing or improving dry skin, preventing or improving oily skin, moisturizing, anti-acne, or inhibiting body odor.
